(19) United States
(12) Patent Application Publication
(10) Pub. No.: US 2009/0228089 A1
(43) Pub. Date: Sep. 10, 2009

(54) Full Thickness Porous Stent

(75) Inventor: Josiah Wilcox, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/041,795

(22) Filed: Mar. 4, 2008

(51) Int. Cl. A61F 2/84; A61F 2/82; B05D 3/00; B05D 1/00

(52) U.S. Cl. 623/1.11; 623/1.46; 623/1.42; 427/2.25

(57) ABSTRACT

A system for treating abnormalities of the cardiovascular system includes a full thickness nanoporous stent having a porous region and at least one therapeutic agent disposed within the porous region. One embodiment includes a full thickness porous cobalt-chromium stent formed by removing magnesium or another sacrificial metal from the stent framework. Another embodiment includes a method of manufacturing a cobalt-chromium stent having full thickness porous regions formed by removing magnesium or another sacrificial metal from the cobalt-chromium alloy comprising the stent framework.

### TECHNICAL FIELD

This invention relates generally to medical devices for treating vascular abnormalities, and more particularly to a full thickness nanoporous stent comprising a cobalt and chromium alloy.

### BACKGROUND

Stents are generally cylindrical-shaped devices that are radially expandable to hold open a segment of a vessel or other anatomical lumen after implantation into the body lumen.

Various types of stents are in use, including expandable and self-expanding stents. Expandable stents generally are conveyed to the area to be treated on balloon catheters or other expandable devices. For insertion into the body, the stent is positioned in a compressed configuration on the delivery device. For example, the stent may be crimped onto a balloon that is folded or otherwise wrapped about the distal portion of a catheter body that is part of the delivery device. After the stent is positioned across the lesion, it is expanded by the delivery device, causing the diameter of the stent to expand. For a self-expanding stent, commonly a sheath is retracted, allowing the stent to expand.

Stents are used in conjunction with balloon catheters in a variety of medical therapeutic applications, including intravascular angioplasty to treat a lesion such as plaque or thrombus. For example, a balloon catheter device is inflated during percutaneous transluminal coronary angioplasty (PTCA) to dilate a stenotic blood vessel. When inflated, the pressurized balloon exerts a compressive force on the lesion, thereby increasing the inner diameter of the affected vessel. The increased interior vessel diameter facilitates improved blood flow. Soon after the procedure, however, a significant proportion of treated vessels restenose.

To reduce restenosis, stents, constructed of metals or polymers, are implanted within the vessel to maintain lumen size. The stent is sufficiently longitudinally flexible so that it can be transported through the cardiovascular system. In addition, the stent requires sufficient radial strength to enable it to act as a scaffold and support the lumen wall in a circular, open configuration

Stent insertion may cause undesirable reactions such as inflammation resulting from a foreign body reaction, infection, thrombosis, and proliferation of cell growth that occludes the blood vessel. Stents capable of delivering one or more therapeutic agents have been used to treat the damaged vessel and reduce the incidence of deleterious conditions including thrombosis and restenosis.

Polymer coatings applied to the surface of the stents have been used to deliver drugs or other therapeutic agents at the placement site of the stent. However, some polymers have been found to be irritating to the tissues they contact during long term implantation. In addition, some biodegradable polymers generate acidic byproducts and degradation products that elicit an inflammatory response.

It would be desirable to provide an implantable therapeutic agent eluting stent without a polymer coating that is capable of releasing one or more therapeutic agents at a therapeutically efficacious rate. Such a stent would overcome many of the limitations and disadvantages in the devices described above.

### SUMMARY OF THE INVENTION

A first aspect of the invention provides a system for treating a vascular condition that includes a catheter, a cobalt-chromium stent having a porous region, the stent being disposed on the catheter, and at least one therapeutic agent disposed within the porous region of the stent. The porous region of the stent framework is formed by removing a sacrificial metal from the cobalt-chromium alloy.

Another aspect of the invention provides a cobalt-chromium stent having a porous region that is formed by removing a sacrificial metal from the cobalt-chromium alloy. At least one therapeutic agent is disposed within the porous region of the stent.

A third aspect of the invention provides a method of manufacturing a therapeutic agent carrying stent that includes providing a cobalt-chromium wire containing a sacrificial metal and leaching the sacrificial metal from the stent framework to form at least one porous region. The method further includes forming a stent framework from the wire, and finally, disposing one of more therapeutic agents within the porous region of the stent.

The present invention is illustrated by the accompanying drawings of various embodiments and the detailed description given below. The drawings should not be taken to limit the invention to the specific embodiments, but are for explanation and understanding. The detailed description and drawings are merely illustrative of the invention rather than limiting, the scope of the invention being defined by the appended claims and equivalents thereof. The drawings are not to scale. The foregoing aspects and other attendant advantages of the present invention will become more readily appreciated by the detailed description taken in conjunction with the accompanying drawings.

### BRIEF DESCRIPTION OF THE DRAWINGS

FIG. 1 is a schematic illustration of a system for treating a vascular condition comprising a porous cobalt-chromium stent coupled to a catheter, in accordance with one embodiment of the current invention;

FIG. 2 is a cross-sectional perspective view of a porous cobalt-chromium alloy stent framework, in accordance with one embodiment of the current invention;

FIG. 3 is a schematic illustration of a portion of porous stent framework having pores of variable density along the length of the stent framework in accordance with the present invention;

FIG. 4a is a schematic illustration of a portion of a porous stent framework having a plurality of pores in the strut portion, in accordance with the present invention;

FIG. 4b is a schematic illustration of a cross section of a porous stent framework, in accordance with the present invention; and

FIG. 5 is a flow diagram of a method of manufacturing a porous cobalt-chromium stent, in accordance with the present invention.

### DETAILED DESCRIPTION

The present invention is directed to a system for treating or preventing abnormalities of the cardiovascular system, cerebrovascular system, urogenital system, biliary conduits, abdominal passageways and other biological vessels within the body. The system comprises a catheter and a porous cobalt-chromium stent disposed on the catheter having a therapeutic agent disposed within a porous region of the stent. After placement of the stent, a therapeutically effective amount of the therapeutic agent is released at the treatment site.

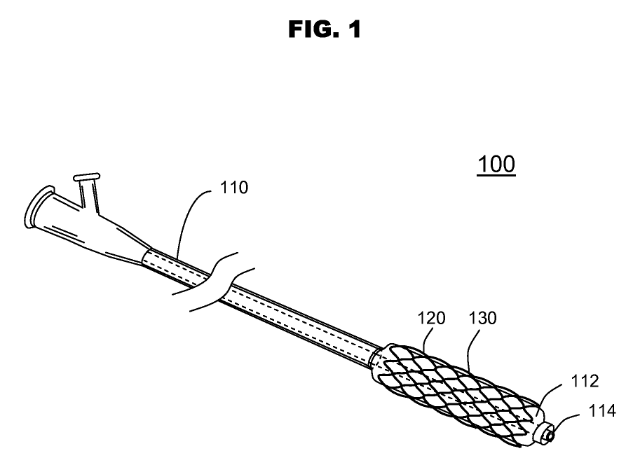

In an exemplary embodiment of the invention, FIG. 1 shows an illustration of a system 100 comprising porous cobalt-chromium stent 120 coupled to catheter 110. Catheter 110 includes balloon 112 that expands and deploys therapeutic agent carrying stent 120 within a vessel of the body. After positioning therapeutic agent carrying stent 120 within the vessel with the assistance of a guide wire traversing through guide wire lumen 114 inside catheter 110, balloon 112 is inflated by pressurizing a fluid such as a contrast fluid or saline solution that fills a lumen inside catheter 110 and balloon 112. Porous stent 120 is expanded until a desired diameter is reached; then the contrast fluid is depressurized or pumped out, separating balloon 112 from porous stent 120 and leaving stent 120 deployed in the vessel of the body. Alternately, catheter 110 may include a sheath that retracts to allow expansion of a self-expanding embodiment of stent 120. Porous stent 120 further comprises a stent framework 130.

In one embodiment of the invention, the stent framework comprises one or more of a variety of biocompatible cobalt-chromium alloys such as MP35N and L605. The cobalt-chromium alloy gives the stent framework the mechanical strength to support the lumen wall of the vessel, while maintaining sufficient longitudinal flexibility so that it can be transported through the cardiovascular system.

The stent framework is formed from a wire or sheet of metallic alloy comprising chromium, cobalt, and a sacrificial metal. In one embodiment, the sacrificial metal is magnesium. The porous region of the wire or sheet of metallic alloy is formed by removal of magnesium by an appropriate dealloying process. The concentration of magnesium will determine the morphology of the porous region including pore size and the degree of porosity. In one embodiment, the magnesium concentration is between 10 and 50 percent of the metallic alloy. In one embodiment, the magnesium concentration is evenly distributed throughout the stent framework. In another embodiment, the concentration of magnesium varies throughout the stent framework. A stent framework having variable magnesium concentration may be formed, for example, by co-extruding alloys having differing magnesium concentration.

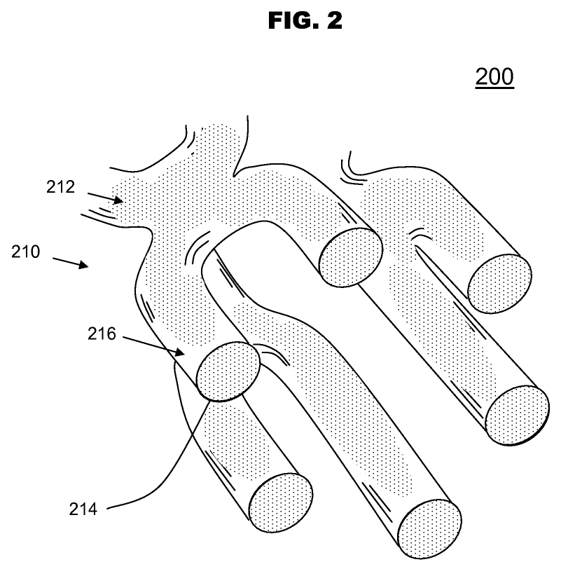

The porous region of the stent comprises a portion of the stent framework having small voids, holes or pores formed therein. The pores are of any appropriate diameter, of uniform or variable size, and may range in size from nanopores to micropores. In one embodiment the pores are nanopores having a diameter between 5 and 120 nm. The degree of density, tortuosity, and depth of the pores in the porous region will depend on the distribution of magnesium in the metallic alloy. In one embodiment, the porous region includes the entire body of the stent framework. In this embodiment, the magnesium is evenly distributed throughout the metallic alloy. When the magnesium is removed by an appropriate dealloying process, the porous region is evenly distributed throughout the structure of the metallic alloy, as illustrated in FIG. 2.

Stent 200 comprises porous stent framework 210 having pores 212 evenly distributed throughout stent framework 210. The pores traverse the entire thickness 214 of stent framework 210. In one embodiment, a thin coating 216 is disposed over the exterior surface of stent framework 210. In one embodiment, coating 216 may be a polymer coating. In this embodiment, polymer coating 216 may be either biostable or biodegradable. In another embodiment the coating includes non-polymeric materials such as dextran, sugars and oils to modify the properties of the coating. In one embodiment, the coating is disposed on the surface of stent framework 210 to modify the rate of therapeutic agent release from stent framework 210.

In another embodiment, the porous regions comprise portions of the stent framework, separated by nonporous regions. In this embodiment, the concentration of magnesium varies throughout the metallic alloy and forms regions of high and low magnesium concentration. Removal of magnesium from regions having high magnesium concentration results in a high degree of porosity, and similarly, removal of magnesium from regions having low magnesium concentration produces regions of limited porosity resulting in a pore distribution of variable density.

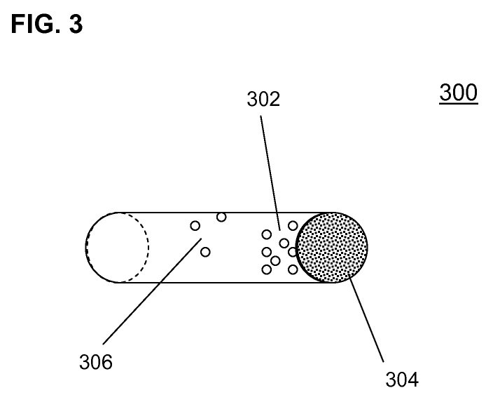

Portion 300 of a porous stent framework having one such distribution of pores is shown in FIG. 3. Region 302 has a high density of pores, extending through the thickness 304 of the stent framework and forming a highly porous structure. In contrast, region 306 has a low density of pores forming a comparatively low porosity structure.

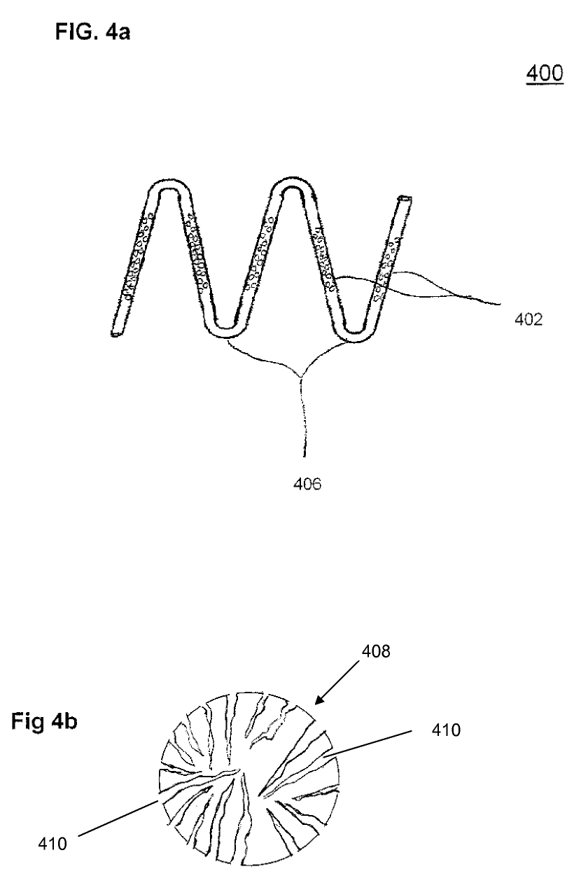

FIG. 4a illustrates another embodiment of a stent framework having areas of variable pore density. Stent framework 400 has been formed so that areas of high pore density 402 form the struts and areas of low pore density 406 form the crowns of the stent framework. This configuration provides low porosity crown portions with sufficient strength to prevent the crown portions from breaking when subjected to strain during expansion and contraction of stent framework. Highly porous areas 402 located on the strut portions of the stent framework are subjected to little strain during stent expansion and contraction and provide pores for therapeutic agent delivery.

FIG. 4b is an illustration of a cross section 408 of high pore density area 402 of stent framework 400. Pores 410 extend from the surface into the interior of stent framework 400, forming a highly porous structure throughout the full thickness of stent framework 400.

In one embodiment, the magnesium is removed from the cobalt-chromium alloy by a chemical dealloying process that removes the magnesium, but leaves the cobalt and chromium structure intact. In one embodiment, the dealloying process comprises exposure of the metallic alloy to nitric acid, sodium hydroxide, or other appropriate dealloying agent. The rate and degree of dealloying will depend on the temperature and time of exposure to the chemical dealloying agent. In one embodiment, the dealloying process comprises exposing the metallic alloy to a 50% nitric acid solution, maintained at 140 C for two hours. The dealloying process may be further modified by applying a voltage, sonic energy or other energy source.

In one embodiment, the dealloying process includes annealing with heat to remove the magnesium and modify the pore size. The annealing process is performed under conditions of appropriate temperature, duration and atmosphere followed by slow cooling. In one embodiment, the alloy is heated to a temperature that exceeds the vapor pressure of magnesium, and is lower than the melting point of the cobalt-chromium alloy. In one embodiment, the cobalt-chromium-magnesium alloy is heated to approximately 600 C for 10 minutes, causing the magnesium to be extruded and a porous cobalt-chromium structure to remain. In some embodiments the annealing process is conducted in a vacuum or under an inert atmosphere. In one embodiment, the pore size is adjusted by heating sufficiently to cause migration or clumping of the cobalt and chromium atoms. In one embodiment the pores thus formed are nanopores.

After the magnesium or other sacrificial metal is removed, the stent framework is formed by shaping the porous cobalt-chromium wire into a cylindrical form. Alternatively, a porous sheet of cobalt-chromium alloy is laser cut and rolled into a tubular shape to form the stent framework. In an alternative embodiment, a nonporous sheet of magnesium-chromium-cobalt alloy is first laser cut and formed into the stent framework, and then dealloyed to remove the magnesium and form the porous regions. In either process, the surface of the stent framework is next cleaned by washing with surfactants to remove oils, mechanical polishing, electropolishing, etching with acid or base, or any other effective process.

In one embodiment, the porous regions of the stent are filled with one or more therapeutic agents. Various therapeutic agents, such as anticoagulants, anti-inflammatories, fibrinolytics, antiproliferatives, antibiotics, therapeutic proteins or peptides, recombinant DNA products, or other bioactive agents, diagnostic agents, radioactive isotopes, or radiopaque substances may be used, depending on the anticipated needs of the targeted patient population. In one embodiment the therapeutic agent is an antiproliferative such as rapamycin, zotarolimus, or an analogue thereof, various inhibitors of the mammalian target of rapamycin (mTOR), and FXB binding drugs. The formulation containing the therapeutic agent may additionally contain excipients including solvents, surfactants, or other solubilizers, stabilizers, suspending agents, antioxidants, and preservatives, as needed to deliver an effective dose of the therapeutic agent to the treatment site. In some embodiments, the formulation is applied as a liquid to the porous zone of the stent framework so that the porous structures are filled with the formulation. The application process may include elevated pressure or vacuum to infuse the therapeutic agent formulation into the porous structure of the stent framework. In one embodiment, the stent framework with the formulation is then dried to remove the solvent using air, vacuum, or heat, and any other effective means of causing the formulation to adhere to the stent framework within the porous structure of the framework. Because the porous structures penetrate the full thickness of the stent framework, the porous structures provide more interstitial space and longer diffusion paths, and therefore can deliver proportionately more therapeutic agent over an extended period of time than porous stents having pores that penetrate only an outer layer or portion of the stent framework.

After delivery of the drug loaded stent to the treatment site, the therapeutic agent will diffuse out of the porous regions of the stent over a defined period of time leaving the porous cobalt-chromium stent in place. A porous nanosurface facilitates covering of the stent by an endothelial cell layer. Additionally, tissue ingrowth into the porous surface of the stent framework may occur. Such tissue ingrowth supports the stent structure and holds the stent in place. Tissue ingrowth into stents and other medical implants is known in the art to provide the advantage of reducing inflammation and foreign body reactions to the implant.

In one embodiment, after the therapeutic agent has been disposed within the porous region, the stent framework is coated with a biocompatible, biodegradable polymer coating such as starch, sugar, dextran, cellulose, polylactic acid, polyglycolic acid, or their copolymers, caproic acid, polyethylene glycol, polyanhydrides, polyacetates, polycaprolactones, poly(orthoesters), polyamides, polyurethanes and other suitable polymers. Such a coating prevents loss of the therapeutic agent through the pores during handling and delivery of the stent and provides a means of regulating the onset of therapeutic agent delivery after placement of the stent. Once in place at the treatment site, the polymeric coating degrades and allows delivery of the therapeutic agent from the porous region.

In another embodiment, the stent framework is coated with a thin porous coating comprising one or more biocompatible, biostable polymers such as polyethylene, polypropylene, polymethyl methacrylate, polyamides, polytetrafluoroethylene (PTFE), polyvinyl alcohol, and other suitable polymers. As the therapeutic agent molecules are released from the porous stent framework, they diffuse through the porous coating to the treatment site. The length of the diffusion pathway thus provided depends on the thickness of the coating, and determines the elution time for the therapeutic agent.

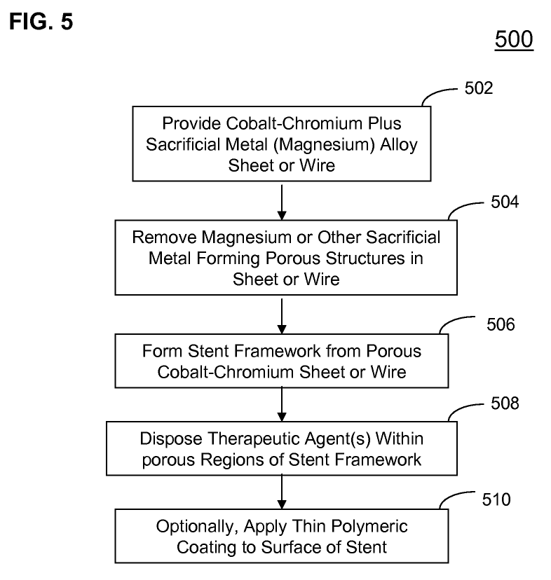

FIG. 5 is a flow diagram of a method 500 of manufacturing a porous cobalt-chromium stent in accordance with the present invention. A cobalt-chromium alloy containing a sacrificial metal such as magnesium is provided in the form of a wire or sheet, as indicated in Block 502. As indicated above, the magnesium content may be either uniformly distributed or dispersed variably throughout the alloy.

Next, the magnesium is removed from the alloy leaving a porous cobalt-chromium metallic wire or sheet, as indicated in Block 504. The magnesium is removed by chemical leaching, heat annealing or any other appropriate means. In one embodiment, the magnesium is leached chemically from the alloy, and then the porous cobalt-chromium structure is subjected to heat annealing to adjust the pore size and modify the properties of the metallic alloy. Using any of the above methods alone or in combination, a porous cobalt-chromium wire or sheet is formed in which the porous structures are nanopores that penetrate the full thickness of the alloy.

Next, as indicated in Block 506, the stent framework is formed from the porous chromium-cobalt wire or sheet. In some embodiments, a porous cobalt-chromium wire is formed into a tubular shape about a mandrel. Alternatively, a porous cobalt-chromium sheet is laser cut and rolled into a tubular shape to form the stent framework.

Next, as indicated in Block 508, one or more therapeutic agent is disposed within the porous regions of the stent framework. In one embodiment, a liquid formulation containing the therapeutic agent(s) is prepared and infused under vacuum into the porous structures of the stent framework. The formulation is then dried to remove the excess solvent using air, vacuum, or heat, and any other effective means of causing the formulation to adhere to the interstitial structures of the porous stent framework.

Finally, in one embodiment, a thin coating is applied to the surface of the stent, as indicated in Block 510. The coating may be biodegradable, in which case it protects the therapeutic agent during handling and delivery of the stent and may additionally provide a smooth surface to facilitate stent delivery. Alternatively, the coating may be biostable, and remain on the stent surface. In this embodiment, the thickness of the coating is selected to extend the time period of therapeutic agent delivery as desired.

The completed stent may then be compressed and mounted on a catheter, expanded at the delivery site, and otherwise handled as needed with minimal loss of the therapeutic agent(s) due to either chemical decomposition or chipping and loss from the stent surface.

In another embodiment a thin, bioabsorbable coating is applied to the external surface of the stent after it is crimped to the balloon portion of the catheter. The coating may be applied using any appropriate technique such as spraying, dipping, vacuum deposition or the like. The coating prevents loss of therapeutic agent during handling and delivery to the active site.

While the invention has been described with reference to particular embodiments, it will be understood by one skilled in the art that variations and modifications may be made in form and detail without departing from the spirit and scope of the invention.

1. A system for treating a vascular condition comprising:
a catheter;
a cobalt chromium alloy stent having a porous region, the cobalt chromium alloy stent disposed on the catheter, the cobalt chromium stent having a stent framework formed by removing a sacrificial metal from the cobalt chromium alloy; and
at least one therapeutic agent disposed within a plurality of pores in the porous region of the cobalt chromium alloy stent.

2. The system of claim 1 wherein the porous region comprises a plurality of nanopores.

3. The system of claim 1 wherein the pores penetrate the full thickness of the stent framework.

4. The system of claim 1 wherein the sacrificial metal is magnesium.

5. The system of claim 1 wherein the distribution of pores along the length of the stent framework is uniform.

6. The system of claim 1 wherein the distribution of pores along the length of the stent framework is variable.

7. The system of claim 1 further comprising a polymeric coating on the exterior surface of the stent.

8. A cobalt-chromium alloy stent comprising a porous region and at least one therapeutic agent disposed within a plurality of pores of the porous region, wherein the porous region is formed by removing a sacrificial metal from the cobalt chromium alloy.

9. The stent of claim 8 wherein the plurality of pores are nanopores.

10. The stent of claim 8 wherein the sacrificial metal is magnesium.

11. The stent of claim 8 wherein the distribution of pores along the length of the stent framework is uniform.

12. The stent of claim 8 wherein the distribution of pores along the length of the stent framework is variable.

13. The stent of claim 8 further comprising a polymeric coating disposed on the exterior surface of the stent.

14. A method of manufacturing a porous cobalt-chromium alloy stent comprising:
providing a cobalt-chromium alloy wire containing a sacrificial metal;
leaching the sacrificial metal from the stent framework to form at least one porous region;
forming a stent framework from the wire; and
disposing one or more therapeutic agents within the at least one porous region.

15. The method of claim 14 wherein the sacrificial metal is magnesium.

16. The method of claim 15 wherein the magnesium is removed from the stent framework chemically or by heat annealing.

17. The method of claim 14 wherein the pores penetrate the full thickness of the stent framework.

18. The method of claim 14 wherein the distribution of pores along the length of the stent framework is uniform.

19. The method of claim 14 wherein the distribution of pores along the length of the stent framework is varied.

20. The method of claim 14 further comprising applying a polymeric coating to the surface of the stent.
